(19) United States
(12) Patent Application Publication
(10) Pub. No.: US 2009/0215738 A1
(43) Pub. Date: Aug. 27, 2009

(54) PREVENTION AND TREATMENT OF AN INCREASE IN CARDIOVASCULAR RISK, ADVERSE CARDIOVASCULAR EVENTS, INFERTILITY, AND ADVERSE PSYCHOLOGICAL EFFECTS AFTER ANDROGEN OR GNRH ANALOGUE INTAKE

(76) Inventor: Michael Charles Scally, Houston, TX (US)

Correspondence Address: ERIC P. MIRABEL, 35 TECHNOLOGY DRIVE, SUITE 100, WARREN, NJ, 07059, US

(21) Appl. No.: 12/391,318

(22) Filed: Feb. 24, 2009

Related U.S. Application Data
(60) Provisional application No. 61/031,842, filed on Feb. 27, 2008.
(60) Provisional application No. 61/031,461, filed on Feb. 26, 2008.

(51) Int. Cl. A61K 31/566; A61K 31/137; A61K 31/138; A61K 31/4196

(52) U.S. Cl. 514/178; 514/651; 514/383; 514/177

(57) ABSTRACT

Disclosed is a is a treatment for the adverse psychological effects (including depression, low self esteem, guilt, increased stress, anhedonia, decreased cognition, sleep disturbances, general fatigue, agitation/motor dyskinesia and decreased appetite) resulting from androgens or GnRH analogue intake, by administering a compound which antagonizes estradiol or its receptors, or blocks and prevents estradiol binding to the estradiol receptors (including antiestrogens) and/or a compound which inhibits endogenous production of estradiol, including aromatase inhibitors. Suitable antiestrogens include clomiphene and its isomer enclomiphene, tamoxifen, 4-hydroxytamoxifen, and toremifene. Suitable cytochrome P450 (P450arom) aromatase inhibitors include formestane, exemestane, anastrozole, and letrozole.

### CROSS-REFERENCE TO RELATED APPLICATIONS

This application claims the benefit of Provisional Application No. 61/031,842 filed on Feb. 27, 2008 and Provisional Application No. 61/031,461 filed Feb. 26, 2008.

### BACKGROUND OF THE INVENTION

1. Field of the Invention

This invention relates to treating an increase in cardiovascular risk, adverse cardiovascular events, infertility, and adverse psychological effects from induced hypogonadism after androgen or gonadotropin releasing hormone (GnRH) analogue intake.

The hypothalamic pituitary testicular axis (HPTA) is the homeostatic system responsible for maintaining, supporting, and ensuring reproduction, bone density, muscle mass, and other important and vital physiological and psychological processes, and is maintained through active in vivo monitoring of hormone levels and feedback.

Structural components of the HPTA are the hypothalamo-pituitary, testicles, and androgen receptor (AR) located on certain end organs (prostate, bone, and muscle). The major hormones monitored by the hypothalamic pituitary testicular axis are gonadotropin releasing hormone (GnRH), luteinizing hormone (LH), follicle-stimulating hormone (FSH), inhibin, testosterone, dihydrotestosterone (DHT), and estradiol. There must be a level of communication between the hypothalamo-pituitary, testes, and androgen receptor (AR) to maintain HPTA homeostasis. The HPTA has two components, both spermatogenesis and testosterone production. They are not equivalent and, in fact, have two very separate hormonal processes for homeostasis. Absent FSH, there is no testicular spermatozoa production. Absent LH, there is no testicular testosterone production.

In males, the pulsatile secretion of gonadotropin releasing hormone (GnRH) from the hypothalamus stimulates LH and FSH secretion. Luteinizing hormone (LH) secretion by the pituitary positively stimulates testicular testosterone (T) production. Follicle-stimulating hormone (FSH) in the presence of testosterone stimulates testicular spermatogenesis. The secretion of GnRH, FSH, and LH is regulated by negative feedback from endogenous testosterone and estradiol as monitored by the hypothalamo-pituitary.

The regulation of the feedback of luteinizing hormone (LH) and follicle-stimulating hormone (FSH) in men is, in part, under the control of estradiol, which results from the aromatization of testosterone. The biosynthesis of estrogens from C19 steroids is by the enzyme aromatase cytochrome P450. This enzyme converts androstenedione and testosterone to estrone and estradiol, respectively.

Estrogens contribute substantially to the negative feedback regulation of gonadotropin secretion. A great part, if not all, of the inhibitory effect on gonadotropin secretion is mediated by the endogenous conversion of testosterone to estradiol. The restraining action of estrogens on gonadotropin secretion in men is exerted both at the pituitary and at the hypothalamic levels. Estradiol has a much larger, inhibitory effect than testosterone, being 200-fold more effective in suppressing LH secretion.

Hypogonadism is a disturbance of HPTA homeostasis caused by inadequate gonadal function, and manifested by deficiencies in spermatogenesis or the secretion of testosterone. Hypogonadism is defined by either reduced reproductive capacity, infertility, or biochemically, by reduced testosterone levels. Hypogonadism has many causes or etiologies.

Hypogonadism has potentially serious consequences that might include but are not limited to adverse body composition changes (decrease muscle mass and increased adiposity), decreased muscle strength, infertility, bone loss (osteoporosis), increase in cardiovascular risk, mood disturbances (depression, anger, low self esteem, sense of well-being, guilt, increased stress, and anhedonia), sexual dysfunction (decreased libido, decreased spontaneous erections, decreased ejaculate, erection dysfunction, decreased sexual fantasies, and anorgasmia), decreased cognitive testing (memory and concentration), sleep disturbances, and constitutional symptoms (general fatigue, agitation, motor dyskinesia, and decreased appetite).

Use of GnRH analogues result in induced hypogonadism by their effects on gonadotropin levels. A GnRH agonist acts as a potent inhibitor of gonadotropin secretion when given continuously in therapeutic doses. Treatment with gonadotropin-releasing hormone (GnRH) analogues inhibits pituitary secretion of LH and thus testicular production of testosterone. Chronic administration of GnRH agonist results in suppression of testicular steroidogenesis, androgen deprivation therapy (ADT). ADT is a form of therapy in the treatment of prostate cancer that is androgen-sensitive. Prostate cancer is one of the most frequently occurring cancers among men in the United States, with hundreds of thousands of new cases diagnosed each year.

Androgen administration results in a form of induced hypogonadism called functional hypogonadotropic hypogonadism. Androgen-induced hypogonadism (AIH) is the functional incompetence of the testes with subnormal or impaired production of testosterone or spermatozoa due to administration of androgens or anabolic steroids. Nonsteroidal androgen administration is currently in the research and investigational stages. Studies on nonsteroidal androgens indicate that their clinical use will result in androgen-induced hypogonadism by their effects on gonadotropin levels. Androgen administration blocks the production of hypothalamic GnRH, this, in turns, shuts down the LH and FSH secretion by the pituitary. This, in turn, reduces the testosterone production by Leydig cells in the interstitium, which along with reducing intratesticular androgen levels and decreased FSH secretion leads to aspermatogenesis. The neuroendocrine abnormality in AIH must be distinguished from classical forms of GnRH deficiency, idiopathic hypogonadotropic hypogonadism (IHH), where the defect in GnRH secretion is clearly permanent rather than transient. Histology of the testicular tissue indicate a reduction in seminiferous tubular diameter, the arrest of advanced spermatogenesis, decrease in the number of spermatocytes and spermatids, Sertoli cell apoptosis, and a depletion of the number of Leydig cells in the interstitial compartment. These are accompanied by changes in semen parameters and testis atrophy. Androgen induced hypogonadism (AIH) occurs in one-hundred percent of individuals upon AAS cessation. The only variable is the duration and severity of AIH.

Cardiovascular disease is the major cause of death among men worldwide. Sex hormones appear to play a pivotal role in determining cardiovascular risk. Androgens in general and testosterone in particular may have some protective effects on the cardiovascular system through their metabolic and direct effects upon human vasculature.

Published literature demonstrates an association between lower androgenicity and increased cardiovascular risk in men. There is a direct correlation between circulating testosterone concentrations and tissue plasminogen activator activity and an inverse relationship between testosterone and plasminogen activator inhibitor-1 activity, fibrinogen, and other prothrombotic factors, suggesting an antithrombotic effect of testosterone.

Testosterone may be involved directly in the regulation of vascular tone. Testosterone has been shown to dilate coronary, aortic, and brachial vasculature by both endothelial-dependent and independent mechanisms. These observations suggest that testosterone may be an important regulator of vascular compliance in large and medium-sized arteries. Increased vascular stiffness has important hemodynamic consequences, and evidence is mounting that vascular stiffness is an independent marker of cardiovascular risk. Arterial compliance or stiffness is a possible modifiable risk factor for cardiovascular disease. This supports the view that physiological levels of androgens may protect the vasculature.

Treatment with GnRH agonists to produce induced hypogonadism significantly increased fat mass, elevated body mass index, increased body fat deposits, decreased insulin sensitivity, rising insulin concentrations, increased insulin resistance, and diabetes in men with prostate cancer, all of which raise the risk of death from heart disease.

Androgen deprivation therapy (ADT) for males results in a hypogonadal state that may have important effects on the vasculature. The effects of ADT on prostate cancer patients found a rise in the augmentation of central arterial pressure, an increase in aortic stiffness, and a reduction in central arterial compliance suggesting large artery stiffening.

After factoring in other known risks for cardiovascular disease (such as diabetes, hypertension, body mass index and smoking), researchers found that the longer patients received ADT, the sooner they were likely to die from heart disease. In 2007, a published study concludes the use of GnRH induced hypogonadism in elderly men is associated with earlier onset of fatal myocardial infarctions. Case reports include myocardial infarction after stopping nonprescription anabolic steroid use.

Studies on induced hypogonadism in healthy young men and ADT in prostate cancer suggest that short-term hypogonadism is sufficient to precipitate depressive symptoms. There is also a trend toward increased aggression while hypogonadal.

Induced hypogonadism produces clinically and statistically significant decreases in the frequency of sexual desire, sexual fantasies, and intercourse. These men also show a strong trend towards decreased spontaneous erections and a significant decrease in the frequency of masturbation.

The possible role of gonadal steroids in regulating sleep and circadian rhythms in humans has received relatively little attention despite the importance of the topic to several clinical syndromes. Results from induced hypogonadism revealed significant decreases in the percentage and time of stage 4 sleep in the hypogonadal state. In addition, studies describe an association between fatigue (diminished energy) and decreased cognitive abilities (memory and concentration).

Reports conclude that signs and symptoms associated with stopping anabolic steroids include fatigue, depressed mood, restlessness, anhedonia, impaired concentration, increased aggression, anorexia, insomnia, decreased libido, self-image dissatisfaction, androgen desire, headaches, suicidal ideation, and feeling depressed/down/unhappy.

Male hormonal contraception uses exogenously administered androgens to suppress pituitary gonadotropins (LH and FSH). Countless publications study the use of testosterone as a male contraceptive agent. The simplistic reason for this is that exogenous administration will cause HPTA suppression, a decrease of sex hormones that includes endogenous testosterone production and the gonadotropins, both follicle-stimulating hormone (FSH) and luteinizing hormone (LH), and as a result infertility. The absent or decreased testicular testosterone production is replaced by its external administration. The individual does not experience the adverse effects of hypogonadism secondary to decreased serum testosterone because of exogenous testosterone administration. This is an induced state of infertility-related hypogonadism, for the express purpose of contraception.

Birth control studies with testosterone administration in physiological as well as subphysiological doses demonstrate HPTA suppression. Data indicate that a return to subject's own geometric mean baseline sperm concentration extends past one year. Other data available from the development of nandrolone decanoate for male contraception indicate that reversal of effects can also take up to twelve months after discontinuation of the drug. An adverse effect from any future male contraception, however, is continued infertility after cessation of the contraceptive agent. Infertility is also a known adverse effect after cessation of nonprescription anabolic steroid use.

A particular androgen, oxandrolone, is currently FDA approved as adjunctive therapy to promote weight gain after weight loss following extensive surgery, chronic infections, or severe trauma, and in some patients who without definite pathophysiologic reasons fail to gain or to maintain normal weight, to offset the protein catabolism associated with prolonged administration of corticosteroids, and for the relief of the bone pain frequently accompanying osteoporosis.

In addition to those FDA approved indications for oxandrolone, clinical studies utilizing androgens, inclusive of testosterone, have received particular attention with regard to improving body composition and muscle strength in those with chronic illness. Androgen use for these purposes is of a limited duration.

Clinical studies utilizing androgen administration include but are not limited to the elderly (sarcopenia is the loss of muscle mass and muscle strength in aging), end stage renal disease (ESRD) on hemodialysis, chronic obstructive lung disease (COPD), HIV+ males, alcoholic hepatitis, glucocorticoid induced osteoporosis, and others. These studies alone results in a very large population receiving androgen treatment. The translation of the clinical studies to direct patient care easily results in androgen administration to millions of individuals.

The disorders above are associated with a considerable degree of morbidity and mortality. Any additional comorbid disease will undoubtedly lead to additional adverse outcomes, not less. In already compromised individuals, hypogonadism, whether or not induced, is a disease with associated particularly significant adverse events that is clearly such a comorbid condition. Published studies utilizing prescribed androgen therapy do not include the follow-up period after androgen intake. This period might potentially leave them in a state of health worse than when first prescribed androgen.

Government health agencies (as evidenced by the labels and packaging inserts associated with FDA approved androgens) and the medical and research community (as evidenced by a lack of exploratory studies, papers and research grants) do not recognize the period after prescribed androgens to be associated with any clinically significant adverse events.

Antiestrogens interfere with the normal negative feedback of estradiol at hypothalamic and pituitary levels in order to increase endogenous gonadotropin-releasing hormone secretion from the hypothalamus and LH secretion directly from the pituitary. In turn, LH stimulates Leydig cells in the testes, and this leads to increased local testosterone production.

Studies demonstrating a significant increase of gonadotropin levels under aromatase inhibition illustrate the important contribution of estrogens to the sex steroid feedback inhibition of gonadotropin secretion in men. Pharmacological inhibition of aromatase activity results in increased levels of gonadotropin and testosterone levels.

Declining, or suppressed, circulating testosterone levels because of induced hypogonadal conditions has many negative consequences in males. There is a direct association between induced hypogonadism (decreased levels of testosterone) and a number of effects, notably an increase in cardiovascular risk, adverse cardiovascular events, infertility, and adverse psychological effects: mood disorders (including depression, low self esteem, guilt, increased stress, and anhedonia), sexual dysfunction (decreased libido, decreased spontaneous erections, decreased ejaculate, erection dysfunction, decreased sexual fantasies, and anorgasmia), decreased cognitive testing (memory and concentration), sleep disturbances, and constitutional symptoms (general fatigue, agitation/motor dyskinesia, and decreased appetite) after androgen or gonadotropin releasing hormone (GnRH) analogue intake. Consequently, there is a need for treatment of these conditions.

### SUMMARY

Disclosed is a treatment for an increase in cardiovascular risk, adverse cardiovascular events, infertility, and adverse psychological effects: mood disorders (including depression, low self esteem, guilt, increased stress, and anhedonia), sexual dysfunction (decreased libido, decreased spontaneous erections, decreased ejaculate, erection dysfunction, decreased sexual fantasies, and anorgasmia), decreased cognitive testing (memory and concentration), sleep disturbances, and constitutional symptoms (general fatigue, agitation/motor dyskinesia, and decreased appetite) after androgen or gonadotropin releasing hormone (GnRH) analogue intake, by administering a compound which antagonizes estradiol or its receptors, or blocks and prevents estradiol binding to the estradiol receptors (including antiestrogens) and/or a compound which inhibits endogenous production of estradiol, including aromatase inhibitors.

Suitable antiestrogens include clomiphene, enclomiphene, tamoxifen, 4-hydroxytamoxifen, and toremifene (as described in U.S. Pat. Nos. 4,696,949 and 5,491,173, which are incorporated herein by reference). Suitable aromatase inhibitors include the steroidal class (type I) of aromatase inhibitors formestane and exemestane and the nonsteroidal class (type II) anastrozole and letrozole. These compounds, or compounds with the same or similar in vivo effects, may be administered separately or in various combinations, at suitable dosages.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### Notation and Nomencalture

Certain terms are used throughout the following description and claims to refer to particular components. This document does not intend to distinguish between components that differ in name but not function.

In the following discussion and in the claims, the terms “including” and “comprising” are used in an open-ended fashion, and thus should be interpreted to mean “including, but not limited to . . . ”.

### DETAILED DESCRIPTION

In an embodiment of a method for treating one or more symptoms after androgen or GnRH analogue intake comprises administering a blocking compound which antagonizes or inhibits estradiol binding to the estradiol receptors and/or an inhibitor compound which inhibits endogenous production of estradiol. Embodiments of the disclosed method may also be used for prevention of an increase in cardiovascular risk, adverse cardiovascular events, infertility, and adverse psychological effects after androgen or GnRH analogue intake.

In some embodiments, the method includes administering one or more androgens, or administering a GnRH analogue to a male subject, and then administering the blocking and/or inhibitor compound after androgen or GnRH analogue intake. More specifically, the blocking compound and/or inhibitor compound may include at least one of an antiestrogen agent, an aromatase inhibitor, or combinations thereof. The one or more effects of induced hypogonadism include an increase in cardiovascular risk, adverse cardiovascular events, infertility, and adverse psychological effects.

As used herein, the term “adverse cardiovascular events” may refer to any unfavorable change in the cardiovascular system of a subject or a patient that may be detrimental to the subject's health. More specifically, an adverse cardiovascular event may comprise myocardial infarction (heart attack), angina, and fatal myocardial infarction.

As used herein, the term an “increase in cardiovascular risk” may refer to any unfavorable change in the cardiovascular system of a subject or a patient that may be detrimental to the subject's health. An increase in cardiovascular risk may comprise the following: elevated serum levels that include but are not limited to the following: triglycerides, homocysteine, lipoprotein(a), prothrombotic factors—fibrinogen, inflammatory markers—C-reactive protein, total cholesterol, LDL cholesterol, insulin, glucose, and tissue plasminogen activator inhibitor-1 activity; decreased serum levels that include but are not limited to the following: HDL cholesterol and plasminogen activator activity. More specifically, an increase in cardiovascular risk may comprise levels or values of the foregoing considered statistically significant within peer-reviewed literature, whether or not at a significance p level of 0.05 or greater.

Furthermore, an increase in aortic stiffness, a reduction in central arterial compliance, and an increase in insulin resistance may adversely affect cardiovascular risk. Insulin resistance is a state in which normal amounts of insulin produce a suboptimal biological response. There are a number of tests used to assess the degree of insulin resistance. The most commonly used tests are the euglycaemic clamp and the homeostatic model assessment (HOMA). The hyperinsulinaemic euglycemic clamp involves simultaneous infusions of insulin and glucose. HOMA is a simpler test and is a mathematical model by which values of insulin sensitivity can be calculated if simultaneous fasting plasma glucose and fasting insulin concentrations are known.

As used herein, the term “adverse psychological effects” includes but is not limited to mood disorders (including depression, low self esteem, guilt, increased stress, and anhedonia), sexual dysfunction (decreased libido, decreased spontaneous erections, decreased ejaculate, erection dysfunction, decreased sexual fantasies, and anorgasmia), decreased cognitive testing (memory and concentration), sleep disturbances, and constitutional symptoms (general fatigue, agitation/motor dyskinesia, and decreased appetite).

A number of methodologies or techniques may be used to evaluate or determine adverse psychological effects including but not limited to: (1) Buss-Durkee Hostility Inventory (a 75-item scale measuring the subscales of assault, indirect hostility [subject is not direct target of hostility], irritability, verbal hostility, guilt, suspicion, resentment, and negativity); (2) Anger, Irritability, and Assault Questionnaire (a 42-item scale assessing variables such as irritability, verbal assault, indirect assault, direct assault, and anger); (3) Barratt Impulsiveness Scale version 7B (a 48-item scale measuring risk taking, interpersonal behavior, motor behavior, self-assessment, and sensory stimulation); (4) Beck Depression Inventory (BDI), a measure of depression severity; (5) Hamilton's Depression Rating Scale; and (6) Spielberger State-Trait Anxiety Inventory, a measure of anxiety severity.

To assess the severity of mood symptoms, the following symptom rating forms may be used, these include but are not limited to: (1) a visual analogue scale; scores range from 0 (symptoms present in the extreme) to 100 (symptoms not present); and (2) the Daily Rating Form (DRF), a 6-point Likert-type scale modified to include the symptoms of interest. Symptoms include but are not limited to the following: avoidance of social activity; loss of enjoyment or interest; impaired function at work or home; irritability or anger; impaired concentration or distractibility; mood swings; feeling depressed, sad, low, or blue; feeling anxious or nervous; decreased eating; increased eating; more sleep, naps, or lying in bed; low energy; loneliness or feeling rejected; feeling physically restless or agitated; feeling powerful, emotionally charged, or pumped up; increased sexual interest; decreased sexual interest; disturbed sleep; drinking alcohol or using nonprescribed drugs; impulse to hurt self; impulse to hurt someone else; acting on impulse to hurt someone; daytime hot flushes; nighttime hot flushes; rapidly changing mood; increased appetite or cravings; a global feeling (best ever/worst ever); low self-esteem; sadness; irritability; low energy; functional impairment; anxiety; and extreme physical discomfort.

A battery of cognitive tests assessing spatial and verbal memory and attention may include but are not limited to the following: Spatial memory measures such as (1) Route Test: This test measures the ability to navigate a short route within a room and (2) Spatial Array Learning Test (SALT): Participants are briefly shown seven unique figures in a particular pattern placed on a grid and asked to recall the correct figure and location. Verbal memory measures such as (1) Proactive Interference: Participants listen to a list of 12 words from the same semantic category (e.g. articles of clothing), and then recall as many of these words as possible and (2) Story Recall: Participants listen to two brief narratives (stories) and asked to recall as much as possible immediately after hearing each story and following a 20-min delay. Divided attention measures such as the Stroop Color Word Interference Task are also used.

Sexual function assessment using an instrument containing items evaluating components of sexual activity, include but are not limited to erectile function, libido, and sexual activity. Because nighttime and spontaneous erections have been shown to be significant clinical indicators, questions used as measure of these aspects of erectile function are whether men reported (yes or no) erections upon waking (“Did you have an erection when you woke up this morning?”) and whether men reported spontaneous erections during the previous 24 h [“Did you have spontaneous erections during the past 24 h (not during intercourse or masturbation, not on awakening from sleep, and not while viewing erotic material)?”]. As a measure of libido, questions allow subjects to circle one of three ratings of their degree of sexual desire, from “no desire at all” to “some desire but not very strong” to “very intense sexual desire,”; masturbation assessment by the question (“Did you masturbate during the past 24 h?”); and sexual intercourse coded as a yes/no response during the previous 24 h [to the question “In the past 24 h, did you have intercourse (penetration with or without orgasm)?”].

A number of methodologies or techniques may be used to evaluate or determine infertility. These include but are not limited to: abnormalities in the number of sperm present, proportion of motile sperm, and morphologically normal sperm; semen collection and analysis of semen volume, sperm density, motility, and morphology according to the World Health Organization (WHO) Laboratory Manual for the Examination of Human Semen Sperm-Cervical Mucus Interaction. Thresholds of <5% normal sperm morphology, a concentration<15×10⁶/ml, and a motility<30% identify the infertile male. These parameters are used in combination to increase the clinical value of semen analysis. Azoospermic specimen contains no sperm, oligospermic specimen reveals concentration of less than 20×10⁶ per ml., and normospermic specimen contains more than 20×10⁶ per ml. Semen analysis is not the sole test for fertility. Fertility determination is a couple-related phenomenon that requires the initiation of a pregnancy.

As used herein, the term “androgen” may refer to any natural or synthetic compound that controls that stimulates or controls the development and maintenance of masculine characteristics in a mammal. The androgen may be steroidal or nonsteroidal. Steroidal androgens include without limitation testosterone, nandrolone, oxandrolone, oxymetholone, stanazolol, dehydroepiandrosterone, androstenediol, androsterone, dihydrotestosterone, or combinations thereof. GnRH refers to GnRH itself or any of its analogues and/or derivatives. The antiestrogen agent and/or aromatase inhibitor may be administered to treat adverse body composition changes or decreased muscle strength after androgen or GnRH analogue intake.

Heretofore, no methods for treating an increase in cardiovascular risk, adverse cardiovascular events, infertility, and adverse psychological effects have been available for patients after medically prescribed or nonprescription use of androgens or GnRH analogues. Accordingly, embodiments of the method may be used after prescribed treatment with androgens and/or GnRH analogues for a condition or disease such as without limitation, human immunodeficiency virus (HIV), prostate cancer, osteoporosis, obesity, sarcopenia, end stage renal disease (ESRD), chronic obstructive lung disease (COPD), alcoholic hepatitis, or combinations thereof.

Without being limited by theory, it is believed that the administration of blocking compounds and/or inhibitor compounds (e.g. antiestrogens or aromatase inhibitors) interferes with the normal negative feedback of estradiol at hypothalamic and pituitary levels in order to increase endogenous gonadotropin-releasing hormone secretion from the hypothalamus and LH secretion directly from the pituitary. In turn, LH stimulates Leydig cells in the testes, and this leads to increased local testosterone production.

As described above, at least one of the blocking compound and/or inhibitor compound is administered to treat an increase in cardiovascular risk, adverse cardiovascular events, infertility, and adverse psychological effects after androgen or GnRH analogue intake.

The blocking compound and/or inhibitor compound (e.g. antiestrogen and/or aromatase inhibitor) may be administered in an amount effective to treat an increase in cardiovascular risk, adverse cardiovascular events, infertility, and adverse psychological effects after androgen or GnRH analogue intake. The blocking compound and/or inhibitor compound may also be administered in an amount effective to reduce the risk of acquiring an increase in cardiovascular risk, adverse cardiovascular events, infertility, and adverse psychological effects after androgen or GnRH analogue intake. Antiestrogen and/or aromatase inhibitors are to be administered in amounts effective to treat adverse effects of androgen or GnRH analogue intake. Dose and duration of medications do not depend on patient weight. Additionally, the treatment dose and duration with antiestrogen or aromatase inhibitor are not affected by the androgen or GnRH analogue type, dose, and duration. The antiestrogen or aromatization inhibitor may be administered once or twice a day. Specific dosages and duration of treatment to maximize efficacy can be derived in human clinical trials or extrapolated from animal studies (based on human/animal weight ratios or other known relationships of the animal model to human dosages).

The antiestrogen(s) may be administered at a daily dosage ranging from 10 mg to about 40 mg, or from about 10 mg to about 80 mg, or from about 25 mg to about 200 mg, or from about 20 mg to about 80 mg. The aromatase inhibitor(s) may be administered at a daily dosage ranging from about 1 mg to about 5 mg, or 0.5 mg to about 2.0 mg, or from about 12.5 mg to about 50 mg, or from about 125 mg to about 500 mg. In addition, the blocking compound and/or inhibitor compound may be administered for a time ranging from about 1 day to about 15 days, alternatively from about 1 day to about 30 days, alternatively from about 1 day to about 45 days, alternatively from about 1 day to about 60 days, alternatively from about 1 day to about 90 days.

In an embodiment, the antiestrogen agent comprises clomiphene, a chemical compound having the formula: 2-[p-(2-chloro-1,2-diphenylvinyl)phenoxy]triethylamine citrate (1:1). In another embodiment, the antiestrogen agent may comprise zuclomiphene or enclomiphene, or a racemic mixture or zuclomiphene and enclomiphene (i.e. clomiphene). In another embodiment, the antiestrogen agent comprises tamoxifen, a chemical compound, which is a triphenylethylene derivative having the formula: (Z)1,2-diphenyl-1-[4-[2-(dimethylamino)ethoxy]phenyl]-1-butene. In another embodiment, the antiestrogen agent comprises 4-hydroxytamoxifen, the active metabolite of tamoxifen. In yet another embodiment, the antiestrogen agent comprises toremifene. More specifically, toremifene is an example of a triphenylalkylene compound described in U.S. Pat. Nos. 4,696,949 and 5,491,173, which are incorporated herein by reference.

Aromatase cytochrome P450 (CYP19) is the enzyme responsible for the synthesis of estrone and estradiol from androstenedione and testosterone, respectively. In an embodiment, the aromatase inhibitor agent comprises the nonsteroidal triazole anastrozole (Arimidex®). In another embodiment, the aromatase inhibitor agent comprises the nonsteroidal triazole letrozole (Femara®). In another embodiment, the aromatase inhibitor agent comprises the steroid formestane (4-hydroxy-4-androstene-3,17-dione). In another embodiment, the aromatase inhibitor agent comprises the steroid exemestane.

Tamoxifen is the trans-isomer of a triphenylethylene derivative. The chemical name is (Z)2-[4-(1,2-diphenyl-1-butenyl)phenoxy]-N,N-dimethylethanamine 2-hydroxy-1,2,3-propanetricarboxylate (1:1). Suitable dosages of tamoxifen range between 10-40 mg per day.

4-hydroxytamoxifen is the active metabolite of tamoxifen. Suitable dosages of 4-hydroxytamoxifen range between 10-40 mg per day.

Clomiphene citrate is 2[p-(2-chloro-1,2-diphenylvinyl)phenoxy]]triethylamine citrate (1:1). Suitable dosages of clomiphene citrate range between 25-200 mg per day.

Clomiphene is a mixture of two geometric isomers which they refer to as cis,-Z-, clomiphene (cis-clomiphene or zuclomiphene) and trans-,E-, clomiphene, (trans-clomiphene or enclomiphene). Suitable dosages of enclomiphene citrate range between 10-100 mg per day.

Toremifene, a triphenylalkylene compound, is a nonsteroidal aromatase inhibitor (inhibitor of estrogen synthesis). Suitable dosages of toremifene range between 20-80 mg per day.

Letrozole is a nonsteroidal aromatase inhibitor (inhibitor of estrogen synthesis). It is chemically described as 4,4′-(1H-1,2,4-Triazol-1-ylmethylene)dibenzonitrile and sold under the trade name Femara. Suitable dosages of letrozole range between 1.0-5.0 mg per day.

Anastrozole is a novel, nonsteroidal aromatase inhibitor [1,3-benzenediacetonitrile,α,α,α′,α′-tetramethyl-5-(1H-1,2,4-triazol-1-ylmethyl)] that blocks the conversion of Δ4-androstenedione to estrone and of testosterone to estradiol. Anastrozole is sole under the trade name Arimidex (anastrozole, AstraZeneca, Wilmington, Del.). Suitable dosages of anastrozole range between 0.5-2.0 mg per day.

Formestane, 4-Hydroxyandrost-4-ene-3,17-dione, is a selective aromatase inhibitor. Suitable dosages of formestane range between 125-500 mg per day.

Exemestane (trade name Aromasin) is an oral steroidal aromatase inhibitor. Suitable dosages of exemestane range between 12.5-50 mg per day.

As an example of a dosing regime, one would administer 50 mg of clomiphene twice a day and 10 mg of tamoxifen twice a day, for a period of 90 days.

It is further contemplated that any analog, derivative, isomer, metabolite, pharmaceutically acceptable salt, pharmaceutical product, hydrate, or N-oxide of the above disclosed antiestrogens and/or aromatase inhibitors may be administered to a patient. Pharmaceutically acceptable salts include without limitation, acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Suitable salts may be formed from the free carboxyl groups and be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administering the disclosed compounds (i.e. antiestrogen and/or aromatase inhibitor) may comprise any methods known to those of skill in the art. More specifically, the disclosed compounds may be administered orally in solid form (i.e. pill) or liquid form (i.e. syrup), or alternatively, may be administered topically through the application to the skin in the form of a cream or lotion. Suitable solid oral formulations include without limitation, tablets, capsules, pills, granules, pellets, etc. Suitable liquid oral formulations include without limitation, solutions, suspensions, dispersions, emulsions, oils, etc. The disclosed compounds (i.e. antiestrogen, aromatase inhibitor) may be part or an ingredient of a pharmaceutical composition such as without limitation, a pellet, a tablet, a capsule, a solution, a suspension, an emulsion, an elixir, a gel, a cream, a suppository or a parenteral formulation. Furthermore, the antiestrogen and/or aromatase inhibitor may be administered intravenously, intraatrially, or intramuscularly in liquid form via a syringe, intravenous line, or other known means.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

Experimental Details Section

### EXAMPLE 1

### Using Enclomiphene to Treat the Adverse Psychological and Behavioral Effects from Induced Hypogonadism After Stopping Prescription Anabolic Steroids in HIV+ Males

Enclomiphene's efficacy in treating the adverse psychological and behavioral effects from induced hypogonadism after stopping prescription anabolic steroids will be verified in HIV+ male subjects currently prescribed androgens for their positive anabolic effects. They will be administered Enclomiphene upon androgen cessation at a dosage of 30 mg, twice per day, for 90 days. Evaluable data is baseline and 90-day psychological testing to include but not limited to sleep disturbance, libido, mood disorder, depression, aggression, and infertility.

### EXAMPLE 2

### Using Enclomiphene to Treat the Adverse Psychological and Behavioral Effects from Induced Hypogonadism After Stopping Nonprescription Anabolic Steroids

Enclomiphene's efficacy in treating the adverse psychological and behavioral effects from induced hypogonadism after stopping nonprescription anabolic steroids will be verified in male subjects currently using nonprescription, illicit, androgens. They will be administered Enclomiphene upon androgen cessation at a dosage of 30 mg, twice per day, for 90 days. Evaluable data is baseline and 90-day psychological testing to include but not limited to sleep disturbance, libido, mood disorder, depression, aggression, and infertility.

The discussion of a reference is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein. The specification and description above are exemplary and not intended to be limiting, and the invention is defined only in the claims which follow and includes all equivalents of the subject matter of the claims.

What is claimed is:

1. A method of treating, preventing, or reducing an increase in cardiovascular risk, adverse cardiovascular events, infertility, and adverse psychological effects from induced hypogonadism after androgen or gonadotropin releasing hormone (GnRH) analogue intake, comprising:
administering to a subject at least one of a blocking compound which antagonizes or inhibits estradiol binding to estradiol receptors, and an inhibitor compound which inhibits endogenous production of estradiol.

2. The method of claim 1, wherein the increase in cardiovascular risk and adverse cardiovascular events are one or more of: fatal myocardial infarction, myocardial infarction, angina, elevated serum levels of one or more of the following: triglycerides, homocysteine, lipoprotein(a), prothrombotic factors—fibrinogen, inflammatory markers—C-reactive protein, total cholesterol, LDL cholesterol, insulin, glucose, and tissue plasminogen activator inhibitor-1 activity; decreased serum levels of one or more of the following: HDL cholesterol and plasminogen activator activity; an increase in aortic stiffness, a reduction in central arterial compliance; and an increase in insulin resistance.

3. The method of claim 1, wherein the adverse psychological effects are mood disorders (including depression, low self esteem, guilt, increased stress, and anhedonia), sexual dysfunction (decreased libido, decreased spontaneous erections, decreased ejaculate, erection dysfunction, decreased sexual fantasies, and anorgasmia), decreased cognitive testing (memory and concentration), sleep disturbances, and constitutional symptoms (general fatigue, agitation/motor dyskinesia, and decreased appetite).

4. The method of claim 1, wherein infertility is <5% normal sperm morphology, a concentration≦15×10⁶/ml, or a motility<30%.

5. The method of claim 1 wherein the blocking compound is an antiestrogen and the inhibitor compound is an aromatase inhibitor.

6. The method according to claim 5, wherein the antiestrogen comprises an analog, a derivative, isomer, metabolite, pharmaceutically acceptable salt, pharmaceutical product, hydrate, N-oxide, or any combination thereof, of the antiestrogen.

7. The method according to claim 5, wherein the aromatase inhibitor comprises an analog, a derivative, an isomer, a metabolite, a pharmaceutically acceptable salt, a pharmaceutical product, a hydrate, an N-oxide, of the aromatase inhibitor.

8. The method according to claim 5, wherein the antiestrogen is selected from the group consisting of clomiphene, enclomiphene, tamoxifen, 4-hydroxytamoxifen, toremifene, and combinations thereof.

9. The method according to claim 5, wherein the aromatase inhibitor is selected from the group consisting of letrozole, anastrozole, formestane(4-hydroxy-4-androstene-3,17-dione), exemestane, and combinations thereof.

10. The method of claim 5, wherein the antiestrogen is clomiphene or enclomiphene administered at a dosage from 10 mg to 200 mg/day.

11. The method of claim 5, wherein the antiestrogen is tamoxifen administered at a dosage from 10 mg to 40 mg/day.

12. The method of claim 5, wherein the antiestrogens are clomiphene and tamoxifen administered at dosages of 50 mg and 10 mg, respectively, twice per day.

13. The method of claim 5, wherein the aromatase inhibitor is anastrozole administered at a dosage of from 0.5 to 2.0 mg per day.

14. The method of claim 5, wherein the aromatase inhibitor is letrozole administered at a dosage of from 1.0 to 5.0 mg per day.

15. The method of claim 5, wherein the aromatase inhibitor is formestane, administered at a dosage of from 125-500 mg per day

16. The method of claim 5, wherein the aromatase inhibitor is exemestane administered at a dosage of from 12.5-50 mg per day.

17. The method according to claim 1, wherein the administering to a male subject at least one of a blocking compound which antagonizes estradiol binding to estradiol receptors or an inhibitor compound which inhibits endogenous production of estradiol comprises intravenously, intraarterially, subcutaneously, or intramuscularly injecting at least one of the blocking compound or the inhibitor compound, or orally ingesting them, or topically applying them.

18. The method according to claim 1, wherein at least one of the blocking compound or the inhibitor compound is administered to the male subject for a period ranging from about 1 day to about 90 days.

19. The method of claim 10 wherein the antiestrogen is enclomiphene administered upon cessation of after androgen or gonadotropin releasing hormone (GnRH) analogue intake at a dosage of 30 mg, twice per day, for 90 days.
